(19) United States
(12) Patent Application Publication
(10) Pub. No.: US 2015/0072929 A1
(43) Pub. Date: Mar. 12, 2015

(54) Pharmaceutical composition comprising a mixture of carboxylated oligopeptides

(71) Applicants: Artur Martynov, Kharkov (UA); Boris S. Farber, Brooklyn, NY (US); Sonya Sophya Farber, New York, NY (US)

(72) Inventors: Artur Martynov, Kharkov (UA); Boris S. Farber, Brooklyn, NY (US); Sonya Sophya Farber, New York, NY (US)

(21) Appl. No.: 14/023,231

(22) Filed: Sep. 10, 2013

(51) Int. Cl. A61K 38/01; A61K 38/28

(52) U.S. Cl. CPC A61K 38/012; A61K 38/28
USPC 514/6.1

(57) ABSTRACT

A pharmaceutical composition comprising a mixture of carboxylated oligopeptides, derived by hydrolysis of eukaryotic or prokaryotic cells and their subsequent partial carboxylation through acylation or alkylation of the amino acid and basic amino acid residues in the amino terminal structure of the oligopeptides. The resulting pharmaceutical composition can be used in production of medical drugs effective in the treatment of cancer, pancreatitis, viral infection, for the development of vaccines.

### CROSS REFERENCE TO RELATED APPLICATIONS

The present application is a continuation-in-part of the applications:

Ser. No. 12/931,467, filed Feb. 1, 2011, which is a continuation of the International Application No. PCT/RU2010/000701, filed Nov. 22, 2010,

Ser. No. 12/931,466, filed Feb. 1, 2011, which is a continuation of the International Application No. PCT/RU2010/000700, filed Nov. 22, 2010,

Ser. No. 12/931,461, filed Feb. 1, 2011, which is a continuation of the International Application No. PCT/RU2010/000692, filed Nov. 22, 2010, and

Ser. No. 12/931,458, filed Feb. 1, 2011, which is a continuation of the International Application No. PCT/RU2010/000690, filed Nov. 22, 2010.

### TECHNICAL FIELD

This invention relates to medicine and pharmaceuticals, specifically, to pharmaceutical compositions and methods of manufacturing pharmaceutical compositions.

### TERMINOLOGY

The term “pharmaceutical composition” means a mixture of carboxylated oligopeptides, derived by hydrolysis of prokaryotic or eukaryotic cells and their subsequent partial carboxylation via acylation or alkylation of the amino acid and basic amino acid residues in the amino terminal structure of the oligopeptides. Also, the composition can include pharmaceutically acceptable auxiliaries such as glycerol, PEG-400, preservatives, stabilizers, cryo- and thermo-protectors.

Application of only one carboxylated oligopeptide of the mixture as a therapeutic or prophylactic agent is not advised, because only in the mixture of many oligopeptides they are capable of forming insoluble stable supramolecular assemblies with each other and with the target.

The term “hydrolysis” refers to the degradation of proteins into smaller fragments of oligomeric oligopeptides in the protease enzyme catalyzed or synthetic analogs of protease. The average size of oligopeptides in this hydrolysis varies from 2 a. r. to 30 a. r. (amino acid residues).

The term “natural polypeptides” refers to the proteins extracted from one or several types of eukaryotic or prokaryotic organisms, such as yeast, bacterial or plant cells, provided by conventional methods, and highly purified of other non-peptide substances—polynucleotides, lipids, polysaccharides and low molecular weight substances.

The term “carboxylation” means introduction of the carboxyl groups into the oligopeptide structure through the formation of a new covalent bond using the method of acylation by dicarboxylic acid anhydrides, or alkylation with alkyl carboxylic acid (No halides). In the case of introduction of polycarboxylic acid anhydrides an amide group is formed from the amino group in the amino acid residues of lysine, histidine, terminal amino acid, and in the case of using alkylcarboxylic acids, alkylamino-derivatives are formed by amino acid residues of lysine, histidine, and end amino groups of oligopeptide.

Partial pattern of carboxylation involves substitution of only part of amino groups in basic amino acid residues (lysine, histidine amino terminal) to oligopeptides. Some residues of lysine, histidine, and the amino end groups remain not substituted. The degree of substitution is calculated based on formulas of combinatorial mathematics to obtain the maximum number of combinations and substitutions either empirically through synthesis of plural derivatives with varying degrees of modification and selection of the most pharmacologically active derivatives.

The term “amino acid residues having free amino groups” means residues of lysine, histidine and amino end groups that are formed during protein hydrolysis with proteolytic enzymes or other form of hydrolysis.

### SUMMARY OF INVENTION

The core of the invention is the pharmaceutical composition that comprises a mixture of carboxylated oligopeptides derived from natural polypeptides by enzymatic or other hydrolysis followed by carboxylation of free amino group of lysine, histidine and of additional end amino groups freed by hydrolysis. Natural polypeptides may be hydrolyzed by proteolytic enzymes, by acid or alkali hydrolysis under mild conditions with formation of oligopeptide mixture. The resulting oligopeptide mixture is further subjected to carboxylation—i.e., acylation or alkylation.

The ratio of modifier to mixtures of oligopeptides is calculated by formulas of combinatorial mathematics in order to obtain the maximum diversity of derivatives in the same volume. Such a system behaves as a quasi-living system and is able to form supramolecular complexes with original proteins, such as between trypsin and acylated oligopeptides of trypsin. The composition may also contain auxiliary substances such as preservatives, stabilizers, plasticizers, and others. The present composition can be used in sterile injectable form, including infusion, or tablets, capsules, suppositories, solution, syrups, ointments, creams, patches, and other pharmaceutical formulations. These formulations can be used as prophylactic and therapeutic drugs: as vaccines in the prevention of infectious diseases, in the treatment of cancer, pancreatitis and other diseases.

### DESCRIPTION OF THE DRAWING

FIG. 1. Black bars illustrates places of insulin hydrolysis, when it is treated with pepsin: only seven peptides are produced, the amino group that should be attacked by anhydride are shown by black arrows (the number of groups available for acylation-n=17).

### DETAILED DESCRIPTION OF THE INVENTION, EXAMPLES

### Example 1

### Quasi-Living Insulin

Diabetes mellitus is one of the most common serious diseases that is based on absolute or relative lack of hormone of the pancreas—insulin.

Therapy by insulin (insulin administration from the outside) is a traditional and single method of treating the disease allowing to compensate for the lack of insulin in the body.

The most common method of insulin administration is by a subcutaneous injection. This method is inconvenient, traumatizing for patients (especially children), causing physical and emotional suffering, but most importantly, it may itself exacerbate the pathology of the disease. The latter is due to the fact that with subcutaneous injection of insulin normal blood glucose levels are achieved through systematic hyperinsulinemia in peripheral tissues, whereas the liver (the main place of activity of the endogenous insulin produced in the body), is lacking insulin.

The only way to prevent the complications inevitably associated with insulin injection, is by achieving whenever possible a complete simulation of the natural pathways of hormone supply in a living organism—i.e., to simulate the physiological difference in the insulin levels in the portal and peripheral circulatory systems.

From this point of view, the oral (by mouth) way of insulin delivery is the most favorable.

The main obstacles hindering the creation of the oral forms of insulin are the hormone low resistance to the action of proteolytic enzymes in the gastrointestinal tract and low permeability of insulin through the epithelial tissue of the intestinal wall into the bloodstream that is due to low lipophilicity, and large size of hormone macromolecules.

Over the past decades there have been numerous attempts to create oral forms of insulin, but no one have succeeded in developing an effective drug that could compete with intravenously injected insulin on the therapeutic action.

Among the pharmaceutical forms of oral medications the most attractive and promising is the solid form, since it is the most comfortable and convenient in application, as well as in storage. In addition, the production technologies of these forms are relatively inexpensive and sufficiently developed.

We have proposed the new kvasi-living self-organizing system for the purpose of creating pharmaceutical oral forms of insulin. The system is a mixture of insulin oligopeptides with artificially increased negative charge of the molecules

The first stage of modification is the enzymatic hydrolysis of insulin molecule (in this case, pepsin). Next, the structure of the synthesized oligopeptides is partially modified in order to replace a part of positive charges in amino groups of lysine and histidine for the carboxyl residues of dicarboxylic acids. Partial modification is actually a combinatorial synthesis that leads to the formation of thousands of different peptides with different structure and specificity. Such a system is protected from the action of intestinal proteolytic enzymes, as it has been already hydrolized, and consists of small oligopeptides. It is freely absorbed from intestine due to small size of its molecules and like a complement can be collected on the insulin receptors into insulin-protein assembly.

Self-assembly of supramolecular peptide systems is also well studied in bacteriophages. Initially, the number of modified peptides is redundant to ensure the process of self-organization in the insulin receptor. If the body of a diabetic has insulin antibodies, or receptors do not match the insulin structure (tolerance to insulin in diabetic type 2); also, if the number of insulin receptors is insufficient the kvasi-living system is capable of self-organization and self-assembly. It automatically picks up from excessive peptides only those components of the “mosaic” that lead to the establishment of a truly effective kvazi-insulin on the receptor. Antibodies do not effect these peptides, since the structure of peptides differs from that of insulin. Small size of the composite oligopeptides and excess negative charge of molecules block generation of antibodies and contribute to the long-term effect of the drug and provides the opportunity to apply such systems orally. Previously, α- and γ-interferons have been also modified by us with the application of kvasi-living systems' technology and they have shown completely new properties.

The purpose of the research was to obtain an oral form of insulin on the basis of kvasi-living self-assembled and self-organized system of acylated peptides derived from enzymatic hydrolyzate of insulin (MI), and to study the effectiveness of the resulting system on the model of alloxan diabetes in rats.

Synthesis of MI on the Basis of Insulin's Succinylated Peptides.

Crystalline insulin (Indar, Ukraine) in the amount of 100 mg was dissolved in 1 ml of 0.1 M hydrochloric acid and then enzymatically hydrolyzed by incubation with pepsin (Fluka, 400 ED/mg) at room temperature for 1 hour. Then, while stirring the solution the powdered succinic anhydride (7.5 mg) was added slowly and incubated with stirring for 60 minutes. The resulting peptides were purified of salts in column Sephadex G-25, with TRIS-hydrochloride as the eluent. The yield of protein was contralled by the absorption of the eluate in the UV region of the spectrum, at 280 nm. Salt-free peptides were poured into vials and lyophilized. Further the hypoglycemic effect of MI on the model of alloxan diabetes was studied in rats: at rest and during glucose load. Input control of insulin was provided using the microfluidic method at bioanalyzer Agilent-2100, chip Protein-80. MI was analyzed using high pressure liquid chromatograph at Millichrom-A-02 (Novosibirsk, Russian Federation) in the Microcolumn, Hypersil-18 at a pressure of 30 kPa 5% ACN, 50 mM ADHP to 60% ACN, 50 mM ADPH. MI was dissolved in 0.9% sodium chloride solution to form the solution, equivalent to 4.3 mg protein/ml.

The Study of Hypoglycemic Action of MI Administered Orally to a Model of Alloxan Diabetes

The experiments used 80 white (albino) rats of Vistar, males weighing 180-220 g. The care of the animals was provided in standard vivarium conditions. were maintained in standard environmental conditions of temperature (22-25° C.), relative humidity (60-70%), dark/light cycle, and fed a standard diet and water ad libitum. All animal procedures were performed according to the Guide for the Care and Use of Laboratory Animals of the National Institutes of Health, as well as guideline of the Animal Welfare Act. Diabetes was induced by single intraperitoneal injections of alloxan monohydrate at a dose of 120 mg/kg, freshly prepared from 0.9% sodium chloride solution. The animals were deprived of food for 24 hours before the injection of alloxan.

Diabetes was fully developed in rats 72 hours after the injection of the toxin, as evidenced by the level of glucose in the blood serum. For the experiments the rats were selected with a fasting glucose content above 11.1 mmol/l (fasting blood glucose). Glucose content in blood, taken from the tail vein was determined using glucometer “One touch Ultra” (USA).

In the first set of experiments, without glucose, the animals were distributed into 4 groups of 10 animals per each:

1—intact control (saline administration);
2—intact rats injected with the modified insulin (MI);
3—diabetes control (infusion of saline solution);
4—animals with diabetes injected with MI.

Rats were fed for 18 hours before and 3 hours after administration of insulin and placebo. Modified insulin was administered in a dose of 50 U/kg, that is 5 times higher than the doze effective in rats (10 U/kg) according to references. The drug was dissolved in saline at the rate of 25 IU/ml and was administered through an intragastric probe in a dose of 0.2 ml/100 g. The control animals were injected saline solution in similar doses. Glucose content in rats blood was assessed prior to drug administration and then in 0.5, 1, 2, 3 and 24 hours thereafter.

In the second experimental setup, with a load of glucose, animals were distributed into 2 groups of 10 animals per each:

1—diabetes control (infusion of saline solution);
2—diabetic animals injected with MI.

The animals were deprived of food for 18 hours before the start of the experiment. Feeding was provided after taking blood samples for the three hour experiment. MI was given per os, in a dose of 50 U/kg, animal control group received saline. After 15 minutes, rats were injected with glucose in a dose of 3 g/kg (40% solution, 0.75 mg/100 g). In the experiments, the drug Glucose was used—the 40% injection solution in vials of 20 ml, manufactured by JSC “Farmak” (Kiev, Ukraine). Immediately before MI injection and 0.5, 1, 2, 3 and 24 hours after the load the glucose content in blood serum was determined.

The research results are processed with the method of variational statistics using Student's test, with significance level P±0.05

The data are presented in Table 1

Results and Discussion

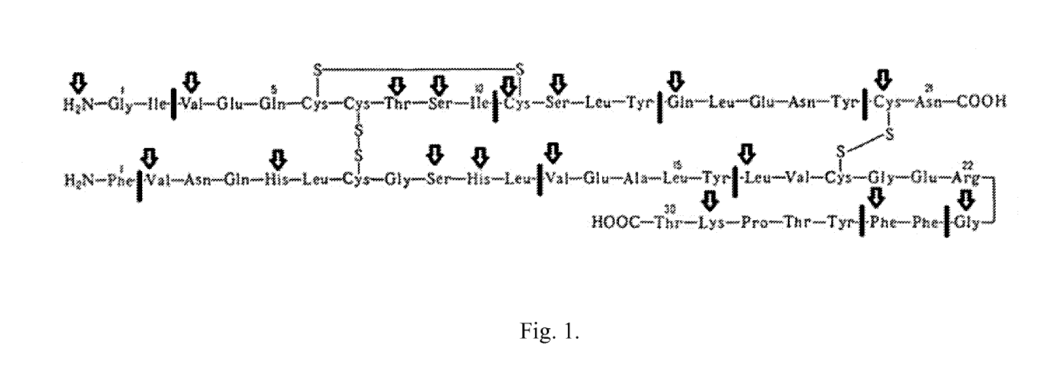

FIG. 1 shows the diagram of insulin enzymatic hydrolysis by pepsin, and places accessible to succinic anhydride attack. As seen in FIG. 1, hydrolysis results in seven oligopeptides. Partial acylation of these peptides is calculated according to the laws of combinatorics to obtain the maximum number of peptide derivatives. The ratio of insulin moles that should be modified to moles of anhydride is calculated according to combinatorial equation:

m=(2n−1), (1)

where:

m-number of molecules (and moles) of insulin, which must be modified to obtain the maximum amount of various insulin derivatives, this value for insulin is equal to 131,071

n-number of amino acid residues available for modification by anhydride in one insulin molecule (it is conditionally accepted that insulin is not hydrolyzed, and represents the whole molecule)

$$k = \frac{n ( {2}^{n} - 1 ) + n}{2} = n 2 ( n - 1 ) \qquad ( 2 )$$

where:

k-number of moles of succinic anhydride, which is necessary for the modification of a protein molecule containing n groups available for modification.

In our case, n=17, k=1114112. Thus, for the modification of 131 071 mol of insulin, 1,114,112 mol of succinic anhydride are required. This results in 131 071 different molecules of succinylated insulin. The molar ratio of anhydride to insulin is 8.5:1. In this case, the synthesis will be observed of the maximum number of different insulin derivatives capable of interaction and self-organization into the supramolecular structure of kvazi-insulin on the insulin receptor.

TABLE 1
Dynamics of glucose content in blood of rats with alloxan diabetes
after single oral administration of insulin peptide supramolecular assembly

| Groups of | Glucose content in blood serum, mmol/l | | | | | |
|---|---|---|---|---|---|---|
| animals | Initial level | 0.5 h | 1 h | 2 h | 3 h | 24 h |
| Control | 4.68 ± 0.20 | 4.76 ± 0.23 | 4.61 ± 0.19 | 4.62 ± 0.15 | 4.51 ± 0.20 | 4.71 ± 0.18 |
| Control + MI | 4.57 ± 0.18 | 4.58 ± 0.19 | 4.67 ± 0.18 | 4.71 ± 0.13 | 4.67 ± 0.13 | 4.56 ± 0.15 |
| Diabetes | 19.48 ± 1.77 | 19.83 ± 1.53 | 18.24 ± 1.34 | 17.58 ± 1.36 | 16.23 ± 1.43 | 19.49 ± 1.29 |
| Diabetes + MI | 18.82 ± 1.00 | 15.50 ± 1.2² | 11.99 ± 1.22^{1,2} | 9.24 ± 1.34^{1,2} | 7.74 ± 1.56^{1,2} | 11.28 ± 1.39^{1,2} |

Notes:
¹Statistically significant differences relative to baseline values;
²Statistically significant differences between groups of Diabetes and Diabetes + MI (p < 0.05).

The modified insulin hypoglycemic action was studied in experiments conducted on rats with alloxan-induced type I diabetes. The results were compared with action of placebo and with intact control. As follows from the data in Table 1, the introduction of the modified insulin into intact animals (without diabetes) did not result in statistically significant changes in blood glucose levels. At the same time the introduction of the modified insulin to diabetic animals caused a significant change in this indicator. In the diabetes control group a gradual slight decrease in blood glucose was observed associated with the lack of food in animals. It is known that in diabetes blood glucose is not a stable and tightly controlled parameter, as it is the case in healthy animals. Within 30 minutes after MI introduction a decrease of glycemia was detected. In all subsequent periods the glucose level decreased, and the differences were significant, both in relation to the original data, and to diabetes control. By 3 o'clock the figures reached almost normal levels and were more than 2 times lower than in the diabetes control group. The rates were significantly lower and 24 hours after MI administration.

The important aspects of MI action are:

1) Gradual pattern of changes, which exclude formation of diabetic hypoglycemia observed with the introduction of injectable forms of insulin. If our hypothesis is correct, this can be explained by the length of the process of insulin molecule self-assembly. This may also explain the lack of glucose reduction in intact animals treated by MI. The duration of the process allows activation of compensatory mechanisms that support stable glucose level in healthy organism (glucagon production, etc.).

2) Exceptional duration of the effect—is up to 24 hours. The phenomenon can be explanated by the following considerations. First, the structure of modified peptides of insulin may differ from the structure of native insulin. This makes them inaccessible to the action of the first enzyme that metabolizes insulin-hepatic glutathione-insulintranshydrogenase that is characterized by a high substrate specificity. Secondly, the modified peptides of insulin, as noted above, may be unresponsive to insulin antibodies—their production does not occur, leaving MI active for a long time.

The action of MI is most clearly manifested under a standard glucose load in the background of fasting for 18 hours. The data show that introduction of MI drastically alters the glycemic curves characteristic of diabetes. There is no distinct increase in glucose level within the first 1-2 hours after the load. The curve in this period is smoothed, and within 3 hours glucose level is reduced to almost normal values. As in the previous experimental setup, 24 hours after administration of the MI blood glucose was also significantly lower than in the diabetes control. Consequently, MI not only reduces glycemia smoothly within 24 hours, but also “takes care” of its postprandial increase.

Thus, MI has the following advantages:

1. mild action, absence of evident hypoglycemia;
2. prolonged effect;
3. smoothing of postprandial hyperglycemia.

Conclusions: The kvasi-living system based on combinatorial acylated derivatives of hydrolyzed insulin has shown high biological activity when administered orally in rats with alloxan diabetes. The system promoted reduction in glucose level to 10 mmol/L on average, and maintained this level within 24 hours after a single application. It can be considered a candidate for development and implementation in the capacity of oral insulin. Efficiency of the preparation was confirmed in animals by using both fasting and glucose load.

### Example 2

### Preparation Anti-Diphtheritic Vaccine Based on the Composition of Vaccine Antigens with Molecules of Changed Charge

Microbial mass is derived from the production strain PW—8 variant Veysenzee by culturing the bacteria C. diphtheriae Linguda in broth with 0.3% glucose or maltose. Culturing is conducted at a temperature of 37° C. for 36 hours; then the microbial mass is separated from toxin by centrifugation (6000 rev/min., for 30 minutes). The resulting sediment (n-gram of fresh weight) is covered with ethanol (concentration 96°) in a volume (2-4) n ml, kept in a refrigerator at 4° C. for 24 hours and then is centrifuged in mentioned mode.

Microbial sediment is covered with (2-10) n ml saline solution, adjust pH to 7.2-7.4 and cooled to 4-6° C. Then it stands for 3-4 hours, and is centrifuged (6000 rpm. for 30 minutes). To the resulting residue is slowly added a solution of 0.8%, disodium ethylenediaminetetraacetate (EDTA-Na2), simultaneously it is triturated in porcelain mortar and to obtain a white, stringy mass. The mass is kept refrigerated at 4°-6° C. for 18 hours. The extract is centrifuged at 6000 rpm within 30 minutes, followed by dialysis against distilled water at pH 7.2-7.5 and is concentrated with polyethylene glycol with molecular weight of 15,000 Da or Superfine Sephadex G 200 to 1 ml volume of n. EDTA extract is re-deposited at pH 7.0.

The extract is then dissolved in phosphate buffered saline containing 0.01% of trypsin, and is incubated at 37 0 C for 12 hours.

The structure of the selected antigen complexes include oligopeptides (75.3±3.4)%, lipids (23.3±4.1)% and carbohydrates (1.4±0.4)%. An aqueous solution of somatic antigens is prepared by focusing on obtaining the necessary concentrations for oral vaccination (5.0 mg/l), is adjusted to pH 8.5-9.0 with 1.0% sodium hydroxide or 1.0% acetic acid, then set in a spectrophotometer (wavelength 230 nm) the exact content of the protein substance. A mixture of antigenic oligopeptides is modified with succinic anhydride relative to the protein content according to the available groups by the formula 1, after setting the number of available groups.

The number of available groups is determined by acid titration after passing the oligopeptide solution through the anion exchanger A2. The optimum weight ratio of the reaction components was—oligopeptides/modifier is equal 3/100. Also for control purposes other complexes were prepared with non-optimal modification degrees for comparison. The immunogenic properties of the complexes were determined in rabbits, chinchilla type, with weight 3.0-3.5 kg. Creation of grund immunity (basic immune reaction to a specific infection) in animals is performed according to the scheme of double antigen injection at a dose of 5.0 mg one hour prior to feeding with daily intervals, for five days. Serological examination is performed 7, 14 and 21 days after the last vaccination using standard diphtheria erythrocytic diagnosticum with a 1:3200 titer (see Table 1).

TABLE 2
Titers of anti-diphtheritic antibodies in sera of rabbits after immunization

| Percentage ratio | Dose of | | | | | | |
|---|---|---|---|---|---|---|---|
| of modifier to | antigen, | Day of | Number of | # of animals with antibodies in titers | | | |
| antigen | mg | observation | animals | 1:10-1:40 | 1:80-1:160 | 1:320-1:640 | 1:1280-1:2560 |
| 1.0% | 5.0 | 7th | 5 | 2 | 0 | 0 | 0 |
| | | 14th | 5 | 2 | 1 | 0 | 0 |
| | | 21st | 5 | 2 | 0 | 0 | 0 |
| 2.0% | 5.0 | 7th | 5 | 0 | 0 | 1 | 4 |
| | | 14th | 5 | 0 | 0 | 3 | 2 |
| | | 21st | 5 | 0 | 1 | 4 | 0 |
| 3.0% | 5.0 | 7th | 5 | 0 | 0 | 2 | 3 |
| | | 14th | 5 | 0 | 0 | 2 | 3 |
| | | 21st | 5 | 0 | 0 | 4 | 1 |
| 4.0% | 5.0 | 7th | 5 | 0 | 1 | 1 | 3 |
| | | 14th | 5 | 0 | 1 | 1 | 3 |
| | | 21st | 5 | 0 | 1 | 2 | 2 |
| 5.0% | 5.0 | 7th | 5 | 3 | 2 | 0 | 0 |
| | | 14th | 5 | 4 | 0 | 0 | 0 |
| | | 21st | 5 | 4 | 0 | 0 | 0 |
| Non-modified | 15.0 | 7th | 10 | 0 | 0 | 0 | 0 |
| antigen | | 14th | 10 | 0 | 0 | 0 | 0 |
| | | 21st | 10 | 0 | 0 | 0 | 0 |

The results indicate that the modified antigens have different ability to influence the humoral immunity. Application of a modifier in an amount of 1.0% or less and 5.0% or more by weight of protein component did not lead to the induction of antibody titers in animals, while acylation of 2.0-4.0% by weight of the protein led to induction of protective titers in the blood of orally vaccinated rabbits to the titer of 1:2560 with keeping it up to 21 days. It is the calculated amount of modifier allowed to obtain the supramolecular complex of oligopeptides to which (when administered orally) the animal organism reacted as to solid un-fragmented antigen and thus these oligopeptides were freely absorbed from the intestine.

### Example 3

### Obtaining Compositions of Modified Peptides (MPS) with Antiviral Properties that are Capable of Self-Organizing into Importins

Under aseptic conditions, 500 mg of ovalbumin is dissolved in 50 ml of distilled water and the pH is brought to 8.0 using 1 M of a sodium hydroxide solution. Trypsin is added, the solution is allowed to sit for 3-45 hours, and the hydrolysis of the ovalbumin with the formation of a peptide mixture is observed. To this mixture, 501-2000 mg of succinic anhydride are mixed for 20 minutes at a temperature of 16-650 C. The mixture is run through membrane filters with the goal of sterilization, and is then poured into glass flagons.

To determine the maximum tolerable concentration (MTC) in the toxological experiments and to study the antiviral activity of the MP drug, the following types of passaged cells of human and animal origin were used:

HS—passaged cells from the kidneys of the embryos of large, horned stock
Tr—passaged cells from the trachea of the embryos of large, horned stock
Hep-2—passaged human larynx cancer cells
Hela—passaged cervical cancer cells
Chicken embryos

The cells were cultured in a 199 medium with the addition of 10% bull blood serum and antibiotics (penicillin and streptomycin). In the capacity of test viruses, the flu virus (H3N2), the vesicular stomatitis virus (Indiana strain), the coronavirus (X 343/44) and the type 1 herpes simplex virus (L-2 strain).

A Study of the Toxicity and Determination of the MTC of the MP Drug on Cell Cultures and Chicken Embryos

To determine the MTC, two-day cultures of cells with well-formed cell monolayers were used. The MP drug was tested five separate times on each of the four types of cells listed above. In each experiment, no fewer than 10 test tubes were used for each of the cultures. After removal of the growth medium from the test tubes, 0.2 ml of the experimental solution and 0.8 ml of support culture medium was added to each test tube. The cells were incubated at a temperature of 370 C over 7-8 days.

Test tubes containing cell cultures to which the drug was not added served as controls.

Calculation of the result was conducted according to the presence or absence of cytopathic activity in the cell when examined under a microscope at ×10. The level of cytotoxic action was determined through changes to the morphology of the cells (cells becoming round or wrinkled, degenerating cells pulling away from the glass) and evaluated according to a four-plus system from + to ++++.

The maximum tolerable concentration was determined by the maximum amount of the substance that could be used without causing cytopathic activity in the cell. For these purposes, various dilutions of the drug at a dosage of 0.2 ml were introduced to the cell cultures.

For a study of toxicity in vivo, the drug was introduced at various doses at a volume of 0.2 ml into the allantoic layer of 9-10-day-old chicken embryos (5 embryos per MP dilution) according to the following method:
10-11-day-old embryos were candled, and a pencil was used to note the location of the air sac on the side opposite to the location of the embryo, where there are fewer blood vessels. The area marked was disinfected with an alcohol and iodine solution; the eggshell was then pierced in that place and 0.1 ml of the material was injected with a tuberculin syringe. In order to reach the allantoic layer, the syringe needle was inserted at a depth of 10-15 mm parallel to the long axis of the egg. After infection, the openings were disinfected again with an alcohol and iodine solution, sealed with paraffin, and placed in an incubator at a temperature of 35-370 C for 72 hours. Before dissection, the embryos were placed for 18-20 hours in a refrigerator at a temperature of 40 C for maximum congealing of the blood vessels. After this, the eggs were placed on a tray blunt end up, the shell over the air sac was disinfected with a solution of iodine and 96% ethyl alcohol; then they were punctured and removed with sterile forceps. The cover over the air sac was also removed after having first separated it from the nearby chorion-allantois membrane. After 24 and 48 hours of incubation at a temperature of 370 C, the number of live and normally developing embryos was counted. The calculations of LD50 and MTD were done according to the Kerber method.

As a result of the study on various cultures, it was established that MPs are non-toxic to cell cultures at a dose of more than 50 mg/ml. (To increase the concentration of the drug, it was lyophilized and then diluted to a concentration of 5%. The results of the toxicity study in various cultures are presented in Table 3.

TABLE 3
The Toxicity of MP in Cell Cultures

| No. | Cell Culture | MTC (mg/ml) |
|---|---|---|
| 1 | Pathogen | more than 50 |
| 2 | Tr | —//— |
| 3 | Hep-2 | —//— |
| 4 | Hela | —//— |

The MTC for cell cultures treated with MP comes to more than 50 mg/ml.

A Study of the Antiviral Activity of the MP Drug on the Influenza a Virus (H3 N2)

Water solutions of MP in various dosages (tenfold dilution) were introduced into 15 chicken embryos in the allantoic layer in a volume of 0.2 ml every 12 hours after introduction of the virus in a working dosage (100 TCD 50/0.2 ml).

Each experiment was accompanied by a control of the test virus in a working dosage. The infected and uninfected (control) embryos were incubated at a temperature of 360 C over 48 hours. Then the embryos from which the allantoic fluid was removed were dissected. The titration of the virus in the allantoic fluid was conducted via the generally accepted methodology with 1% erythrocytes of human blood type 0(1).

The protection factor (PF) was determined in accordance with [1].

The titer of the virus in the experimental and control groups of chicken embryos is presented in Table 4.

TABLE 4
Effective Concentration of MP in
in ovo Influenza Infection Models

| | | | | Minimum |
|---|---|---|---|---|
| | | Virus Titer | | Effective |
| | Drug | (lg TCD 50/ml) | | Concen- |
| | Concentration | Experi- | | tration (MEC |
| Group | (mg/ml) | ment | Control | mg/ml) |
| Control (injected | — | 12 | 12 | — |
| with a 0.9% saline | | | | |
| solution) | | | | |
| Control Group | 50 ± 5 | 0 | 12 | 0.05 |
| | 5 ± 1 | 0 | 12 | |
| | 0.5 ± 0.05 | 2 | 12 | |
| | 0.05 ± 0.005 | 4 | 12 | |
| | 0.005 ± 0.0005 | 10 | 12 | 5 |

As may be seen in Table 4, the minimum effective concentration of MPs in relation to the influenza virus that fully stops viral synthesis is equal to 0.05 mg/ml. When the dilution of the drug is increased, the effectiveness of the MP declines and has a dose-dependent nature. This fact bears witness to the presence of a direct antiviral effect against the H3N2 virus in the MP drug.

Study of the Antiviral Activity of the MP Drug on Cytopathic Viruses (Vesicular Stomatitis Virus, the Coronavirus, and HSV-1)

The antiviral activity in relation to this group of viruses was determined in cultures of the abovementioned cells. The reaction was produced in the following manner: 0.2 ml each of the corresponding virus in a working dosage (100 TCD50/0.2 ml) was introduced into a two-day rinsed cell culture. 0.8 ml of supporting medium was added. When cytopathic activity was observed in the culture, the MP drug was introduced in various doses. As a control, the same test viruses were used without the drug. The cells were incubated at a temperature of 370 C Reports on the experiment were done on the third, fifth, and seventh days.

A decline in the virus titer under the influence of the drug being tested of 2 Ig or more in comparison with the control was determined to indicate antiviral activity.

The results of the study of the antiviral activity of the MP drug are presented in Table 5.

TABLE 5
Study of the Antiviral Activity of the MP Drug on Vesicular
Stomatitis Virus, the Coronavirus, and HSV-1.

| | | MEC, | Maximum Decline in Titer |
|---|---|---|---|
| Drug | Virus | mg/ml | of the Virus, lg TCD 50/ml |
| MP | VVS | 0.05 | 3.8 |
| | CV | 0.05 | 2.8 |
| | HSV-1 | 0.05 | 4.8 |

As may be seen in Table 5, MPs have antiviral activity and ability to stop the reproduction of all viruses we studied at a concentration of 0.05 mg/ml with a MTC of 50 mcg/ml. The drug's CTI is 1000. Moreover, MP was active in relation to all the viruses studied, while not one comparison drug showed the same kind of activity. Thus the drug is not connected with the specific characteristics of the virus or cell culture, but rather affects mechanisms that all cells have in common.

A Study of the Antiviral Activity of MP In Vitro in Models of Farm Animal Viruses

The tests were run on 96-lunula plastic panels with viruses of the transmissible gastroenteritis of swine (TGS), strain D-52, with an initial titer of 104.0 TCD50/ml (tissue cytopathic doses) in a test tube culture of piglet testicle cells (PTC) and the diarrhea virus for large horned stock of the Oregon strain with an initial titer of 1070 TCD50/ml in a test tube culture of saiga kidney cells (SKC).

In a study of virustatic (inhibiting) activity, the cell cultures were infected with the viruses in doses of 100 and 10 TCDunits/ml and incubated at a temperature of 37° C. MPs were introduced in various doses to the cell cultures (CC) 1-1.5 hours after infection (after the absorption period). Eight titer wells were used for each dilution. After introduction of the sister compounds, the cell cultures were incubated at 37° C. for 72-144 hours until clear evidence of cytopathic activity was found in the virus control.

The cell cultures infected by the virus, inactive CCs, and CCs to which only various concentrations of MPs were introduced served as the control groups. The virustatic activity was determined by the difference in the titers of the viruses in the experimental and control groups.

When virucidal (inactivating) activity was determined in various dosage levels of the solution of sister compounds, they were mixed in various amounts with virus-containing materials and incubated at a temperature of 37° C. over a 24-hour period. The control was the virus-containing material, to which, in addition to the solution of the sister compounds was added a placebo (physical solution) and inactive cell cultures. After contact, the mixtures were titered in parallel with the control. The results were calculated 72-144 hours after incubation at 37° C., after an obvious manifestation of cytopathic activity in the control viruses. The virucidal action was determined by the differences in the titers of the experimental and control group viruses and were expressed in Ig TCD50.

As a result of the studies conducted, it was established that an MP compound in a concentration of 4000 mcg/ml stopped the reproduction of the TGS virus at 2.75 Ig TCD50/ml at an infectious dosage of 100 TCD50/ml and in the same does at 3.75 Ig TCDunits/ml at an infectious dosage of 10 TCD50/ml. At a dose of 4000 mcg/mg the TGS virus was inactivated at 2.0 Ig TCD50/ml. The MP compound at a dose of 4000 mcg/ml inactivated the diarrhea virus for large horned stock at 3.5 18 TCD50/ml.

When toxicity was studied, it was discovered that MPs at a dose of 4000 mcg/ml were not toxic to either cell culture.

Thus the MP compounds have virustatic (inhibiting) and virucidal (inactivating) activity on the TGS virus and the diarrhea virus in large horned stock; chemical drugs may be created based on these compounds for the treatment and prevention of infectious illnesses of viral etiology.

A Study of the Antiviral Activity of MP in an Experiment on Animals (Herpesvirus Kerato-Conjunctivitis/Encephalitis in Rabbits)

The specifics of the experimental system and the level of its adequacy against natural human illness undoubtedly play a decisive role in the evaluation of the effect of antiviral substances on the course of an infection. Experimental herpes infections are of interest in that diseases caused by herpes are widespread and extremely variable in clinical symptomology. The models of experimental herpes on animals are finding increasingly wide application in the study of new antiviral substances.

As is well-known, one of the clinical forms of systemic herpes is herpetic encephalitis, which occurs in guinea pigs, hamsters, rats, mice, rabbits, dogs, and monkeys.

Herpetic keratitis/conjunctivitis was caused in rabbits with an average weight of 3.5 kg through introduction of infected material (herpes 1 virus, L-2 strain) into a wounded cornea. The animal was immobilized and its eye was anesthetized with dicaine (eye drops). The eyelids were pulled back, and several scratches were made on the cornea with a syringe needle. Then the virus-containing material was introduced. The eyelids were closed and rubbed in a circular motion against the cornea. Viral dose: 0.05 ml In the experiment, 16 rabbits were used; of these, 10 were given MPs (daily, beginning on the second day of infection; 14 days at a dosage of 21 mg/kg [which is 7.5 ml of a 1% solution per animal per day]), while six were given a placebo (0.9% sodium chloride).

After the rabbits were infected with HSV-1, the condition of their corneas was observed daily for presence of keratoconjunctivitis, encephalitic damage, and presence in the lymphocytes of the peripheral blood of HSV-1 antigens through the immunofluorescence reaction method before and after infection. Before infection, all the animals' lymphocytes were missing the specific luminescence, which indicated that they did not have antigens to the HSV-1 virus in their peripheral blood. On the third day after infection, the blood of all the animals showed an antigen of HSV-1, IF=70%. In addition, three rabbits (two from the experimental group before treatment and one from the control group) showed encephalitis symptomology: convulsive disorder, loss of appetite.

Keratoconjunctivitis developed in all the animals. On the fourth day after infection, the experimental group was administered MPs to the ear vein at a dosage of 21 mg/kg body mass; the control group was administered 0.9% solution of sodium chloride. Over the course of two weeks, this procedure was repeated once a day. In the experimental group, all the animals survived and HSV-1 antigens were not found on the 13th or 14th day. Moreover, in the experimental group, the encephalitis symptoms disappeared by the seventh day of drug administration, whereas in the control group, two animals died. By the 14th day, one animal in the control group had died, while six had died in the control group. Accordingly, the effectiveness indicator was equal to 83.3%, which indicates the high treatment effectiveness of MPs in the model of herpes keratoconjunctivitis/encephalitis in rabbits. In addition, the rabbits in the experimental group gained weight and none showed signs of keratoconjunctivitis. The chemotherapeutic index for rabbits for the MP drug came to 1000, which indicates the promise of MPs as a highly effective antiviral drug with a wide spectrum of activity and low level of toxicity.

Confirmation of Albuvir's Mechanism of Action

To confirm the MP's mechanism of action, we used DNA from the type 1 L-2 herpes viral strain. They were distinguished as indicated in [1]. DNA conjugation with gold particles was conducted according to the method from [2].

These liposomes merged with the cell membranes from the chicken fibroblast culture. After the merging of the liposome with the cell membrane, the virus's DNA entered the cytoplasm along with the gold particles.

The α-β-importin complex carried the colloidal particles into the nuclear pores with the polynucleotide. If the cells were incubated in the presence of the MP, aggregation of the particles of colloidal gold in the nuclear pores was not observed. All the particles were equally distributed throughout the cells' cytoplasm. In this case, the cytopathic activity of the herpes virus was not observed.

Thus the MP slow the process of the transportation of viral DNA to the cell nucleus, which was to be proven.

The Effectiveness of the MP Drug on KO66-500 Cross Chickens

The goal of this experiment was the study of the effect of the MP drug on the reproduction of vaccine strains of viruses in the reduction of the titers of the corresponding specific antibodies. It is known that many antiviral drugs, when stopping the reproduction of the live vaccine strains of the viruses lead to the depression of the synthesis of specific antiviral antibodies. This effect is connected with a shortfall in intensity of the infectious process caused by the vaccine in the birds' bodies, and to a weak immune reaction. It is known that in many cases—for example, in infectious bursal disease—the use of live vaccine leads to the induction of the synthesis of such an excessive antibody titer that the bursa becomes exhausted, the bird becomes sensitive to other viruses, and a decrease in weight and increase in mortality occurs. The application of the MP drug should have indicated that it contained antiviral properties according to several parameters: reduction in the excess level of antibody (titers), a decrease in the mortality rate (preservation), and an increase in weight.

For the experiment, 15 chickens per group were used; each was between 36 and 41 days old. The MPs were applied a day before vaccination with live IBD, Gamboro Disease (GB), and infectious bronchitis (IB) vaccines. In the control group were birds that had not been treated with MPs but had been vaccinated. The results of the study are presented in Tables 6 and 7.

TABLE 6
Weight Gain of Chickens (at Time of Slaughter)
in Experimental and Control Groups

| Indicator | Weight Gain**, +% | Survival**, +% |
|---|---|---|
| Experimental Group | 5.2 ± 0.7* | 1.1 ± 0.3* |
| (n = 15) | | |
| Control Group | −1.2 ± 0.3* | −2.1 ± 0.5* |
| (n = 15) | | |

*against the unvaccinated control, which is taken for the base.
**(P = 0.01)

As may be seen in Table 6, in the experimental group, the animals' weight increased by (5.2±0.7)%, while a decrease in weight of (−1.2±0.3)% was observed in the group that was vaccinated but not treated. Also, an increase in survival rates of (1.1±0.3)% was observed in the experimental group.

In Table 7 is presented the change in the titers of specific antiviral antibodies in the group that was treated with MPs and vaccinated, the group that was vaccinated but not treated, and the group that was not vaccinated.

TABLE 7
Changes in the Titer of Antibodies to Infectious Bursal Disease
(IBD), Gamboro Disease (GD), and Infectious Bronchitis (IB)
in Vaccinated Groups and an Un-Vaccinated Control

| | Average Change in the Titer of | | |
|---|---|---|---|
| | Specific Antibodies, ±T | | |
| | IBD | GD | IB |
| Experimental Group | −1000 ± 400 | −600 ± 200 | −1200 ± 400 |
| (vaccinated and | | | |
| treated with MPs) | | | |
| (n = 15) | | | |
| Control Group No. 1 | +2600 ± 700 | +3200 ± 1200 | +2700 ± 1000 |
| (vaccinated, but not | | | |
| treated with MPs) | | | |
| (n = 15) | | | |

| | |
|---|---|
| Control Group | 0 |
| (not treated or | |
| vaccinated) | |

As may be seen in Table 7, the MP has a direct (not immune stimulating) action against all three viruses. The most inhibiting effect was observed in the group with infectious bronchitis: a reduction in the antibody titer by 1200 units. In the vaccinated but untreated control group, the titers of antibodies grew from 2600 units to 3200 units, which indicated that the process of multiplication of the live vaccine in the birds' bodies had been effective.

Thus the application of MPs allows an average of a 5% weight gain in the chickens and a 1% decrease in mortality.

MPs have a direct antiviral action, which stops the reproduction of the viruses that cause infectious bursal disease, Gamboro Disease, and infectious bronchitis.

MPs allow the reasonable restriction of the replication of the vaccine viruses, facilitating a sufficient level of protective antibodies and preventing the exhaustion of the birds' immune systems and the corresponding decrease in weight and increase in mortality.

The Effect of MPs on the Effectiveness of the Vaccination of Chickens with Live Vaccine

The effect of MPs on the effectiveness of the vaccination was observed directly in an aviaculture business during raising of chickens. When a pathological and anatomical study of the chickens was conducted, characteristic changes were seen for colibacillosis and coccidiosis, as well as many hemorrhages in the mucous membranes of the large intestines and in the transition section between the proventriculus and the gizzard and grinding glands. The contents of the proventriculus were dyed green. The chickens' death rates came to about 15-20% When blood serum of chickens from 38-42 days of age were studied in a hemagglutination inhibition test (HI), specific antibody titers to the Newcastle Disease virus were found that were higher than protective levels (1:1024, 1:2048).

The study of the effect of MPs at a dosage of 0.03 ml/kg of live weight on the effectiveness of the Newcastle Disease vaccine. For this purpose, one of the aviaries was taken as the control; the others were experimental (Table 8).

TABLE 8
Results of the Study of the Effect of MPs on the
Effectiveness of Vaccination in Aviculture

| | | No. of | |
|---|---|---|---|
| | Aviary | Heads | |
| Group No. | No. | (thousands) | MP Dosage Schedule |
| Control | 4 | 40.0 | MP Not Given |
| Experiment 1 | 8 | 40.0 | From an age of seven days over |
| | | | the course of the three days |
| | | | before vaccination with live |
| | | | viral vaccine |
| Experiment 2 | 7 | 40.0 | 1 Day before Newcastle |
| | | | Disease Vaccination |
| Experiment 3 | 5 | 40.0 | Over the 3 Days before |
| | | | Vaccination and 7-10 Days after |
| | | | Newcastle Disease Vaccination |

The conditions of observation, the microclimate parameters, the lighting regime, the amount of floor space per bird, and the feeding schedule were identical throughout all groups in accordance with the methodological recommendations for raising ROS 308 crosses.

The immune system load was determined at an age of 42 days through HI. Simultaneously, the clinical condition of the birds, their retention rate, their growth, and food loss were calculated.

The results of the experiments for the determination of the effectiveness of MPs when vaccinating chickens against Newcastle Disease are presented in Table 9.

TABLE 9
The Effect of MPs on the Effectiveness
of the Newcastle Disease Vaccine

| | | Experiment | Experiment | Experiment |
|---|---|---|---|---|
| Indicators | Control | 1 | 2 | 3 |
| Average | 21 ± 7.55 | 39.0 ± 15.30 | 84.5 ± 29.39 | 124.0 ± 31.09** |
| Titer, | | | | |
| in HI | | | | |
| Immune | 75 | 87.5 | 100 | 100 |
| System | | | | |
| Stress, % | | | | |

Notes:
Reliability in comparison with the control:
*P < 0.05,
**P < 0.01
***P < 0.001

The average titer of specific antibodies to the Newcastle Disease virus were on the protective level in both the control and experimental groups. However, when the 42-day-old chickens' serum was studied, the ones with MPs used established a significant increase in the average titer in experimental group 3 in comparison with the control group by a factor of 6 (<0.01). In the experimental groups (1, 2), a reliable difference in the antibody titers in comparison to the control could not be established; however, they were on the protective levels, and a tendency to increase this indicator by factors of 1.8 and 4.3 was discovered. The group immunity in the control came to 75%, while it was 100% in experimental groups 3 and 4 and 87.5% in another experimental group. The death rate of the chickens in the control group was 9.8%, while the death rates fell in the experimental groups by a factor of 2.8, 3.3, and 4 respectively in comparison with the control. The average daily growth of the chickens in the experimental groups fluctuated from 52-54 g, while the growth in the control group was 48 g.

Thus a conclusion may be drawn that the optimum scheme for the use of MPs for chickens in regions with complex epizootic situations with Newcastle Disease is the use of the drug at a dosage of 0.03 ml/kg of live weight over the course of 3 days before vaccination and 7-10 days after vaccination against Newcastle Disease. The use of the drug according to the abovementioned scheme will lead to an increase in the average titer of specific antibodies to the Newcastle Disease virus by a factor of 6 and a decrease in the death of the chickens by a factor of 4.

### Example 4

### Obtaining the Ologopeptides Composition as the Trypsin Inhibitor

1.0 g trypsin is dissolved in 100 ml distilled water neutralized to pH=7.5 for the creation of a 1% solution; this is left to set for 48 hours at a temperature of 370 C for autolysis. Then 2.0 g succinic anhydride is added to the peptide mixture produced; this is stirred until fully dissolved. The solution of peptides is poured into 5 ml test tubes, lyophilized, and used as a trypsin inhibitor.

A Study of the Anti-Trypsin Activity of the Derived Peptides

To determine the minimum effective concentration of the drug, a twofold dilution was prepared. An effective concentration is a dose of the peptide formula that fully inhibits the trypsin's proteolytic activity. In the capacity of a protein target for the action of the trypsin, 1% sodium caseinate with a phosphate buffer at a pH of 8.0. The concentration of oligopeptides in solution that were products of hydrolysis over time was determined by a spectrophotometer at 280 nm and 260 nm. A trypsin solution was added to a solution of 1% casein at an enzyme:protein ratio of 1:100; every five minutes, 1 ml of sample was taken and the same volume of 1% trichloracetic acid was added. The protein sediment created was centrifuged, and the concentration of dissolved peptides created after hydrolysis was determined by a spectrophotometer. The spectrophotometry method of protein determination is based on the ability of aromatic amino acids (tryptophan, tyrosine, and to a lesser extent, phenylalanine) to absorb ultraviolet light, with the maximum absorption at 280 nm. It is conditionally acceptable to believe that at a protein concentration in the solution equal to 1 mg/ml, the optical density value at 280 nm is equal to 1 when cuvettes with a layer thickness of 10 mm are used. The drug's eluent was used in the capacity of a comparison solution. The concentration of the experimental protein in the solution must be from 0.05 to 2 mg/I. The presence of nucleic acids and nucleotides (more than 20%) inhibit the identification of the protein. In this case, the optical density of the same solution is measured at two wavelengths: 260 and 280 nm; the amount of protein X (mg/ml) is calculated using the Calcar formula:

X=1.45*D₂₈₀−0.74*D₂₆₀

The more dissolved peptides there were in the solution, the more active the trypsin was. Inhibiting trypsin should have led to decreasing the concentration of the dissolved peptides.

The results of the study of the anti-trypsin activity of the peptide formula being patented follow.

TABLE 10
Dependence of Trypsin Activity on the Dilution
of Added Succinylpeptide Inhibitors

| | | | Concentration of |
|---|---|---|---|
| | | | Dissolved Peptides |
| | | | after One Hour of |
| | | Enzyme Activity | Incubation |
| No. | Specimen | (U/ml)* | (mg/ml)* |
| 1 | Trypsin (control) | 0.6 ± 0.05 U/ml | 10 ± 1 |
| 2 | Trypsin + | 0.1 ± 0.05 U/ml | 1.7 ± 0.2 |
| | succinylpeptides in a | | |
| | concentration of 0.125 | | |
| | ng/ml | | |
| 3 | Trypsin + | 0 | 0 |
| | succinylpeptides in a | | |
| | concentration of 0.25 | | |
| | ng/ml | | |
| 4 | Trypsin + | 0 | 0 |
| | succinylpeptides in a | | |
| | concentration of 0.5 | | |
| | ng/ml | | |
| 5 | Trypsin + | 0 | 0 |
| | succinylpeptides in a | | |
| | concentration of 1 | | |
| | ng/ml | | |

*P < 0.01

As may be seen in the table, the effective concentration of succinylpeptides is 0.125 ng/ml at a trypsin concentration of 0.1 mg/ml. The experiment also confirmed the effectiveness and dosage-dependent nature of the formula being applied.

Study of the Effectiveness of the Composition of Peptides in Treating Animals with Severe Pancreatitis

In the study, a model of severe pancreatitis in mice induced by interperitoneal introduction of caerulein was used [3]. The intensity of the pancreatitis was correlated with the concentration of amylase in the blood of the mice. Pancreatitis was induced in mice 16-20 g in weight through intraparenteral introduction of caerulein in a single dose of 100 mcg/kg body weight. Caerulein was introduced again at an interval of six hours. To verify the hypothesis on the drug's effectiveness specifically in the treatment of pancreatitis, the formula of derived peptides were introduced into the animals interparenterally at a dosage of 0.1 ml 1 ng/ml once a day for three days in a row. The concentration of amylase in the animals' blood was verified daily.

TABLE 11
Indicators of the Effectiveness of the Peptide Formula
Being Patented on Pancreatitis Models in Mice

| | Amylase | Number of Mice | Number of Mice |
|---|---|---|---|
| | Concentration | in Experimental | Dead within |
| Specimen | U/ml | Group | 10 Days |
| Caerulein and | 66.4 ± 11.1* | 12 | 6 |
| Physical Solution | | | |
| Caerulein and | 14.1 ± 2.3* | 12 | 0 |
| then the | | | |
| Formula Being | | | |
| Patented | | | |
| Control | 9.2 ± 1.1 | 5 | 0 |
| without | | | |
| Caerulein | | | |

*P < 0.01

As may be seen in the data presented in Table 11, the formula being patented turned out to be capable not only of normalizing the level of amylase in the blood of the animals nearly to the level of that of the control group, but also of preventing their deaths. While there was a 50% mortality rate in the control group, all the animals in the experimental group survived. Thus the formula of peptides being patented had a therapeutic effect on models of severe pancreatitis in mice.

### Example 5

### Obtaining Mixtures of Modified Peptides (MPs) with Anti-Cancer Properties that are Capable of Self-Organizing into Importins

Under aseptic conditions, 500 mg of lactabumin is dissolved in 50 ml of distilled water and the pH is brought to 8.0 using 1 M of a sodium hydroxide solution. Trypsin is added, the solution is allowed to sit for 3-45 hours, and the hydrolysis of the lactalbumin with the formation of a peptide mixture is observed. To this mixture, 501-2000 mg of succinic anhydride are mixed for 20 minutes at a temperature of 16-650 C. The mixture is run through membrane filters with the goal of sterilization, and is then poured into glass flagons.

The anti-cancer activity of MPs. The determination of the anti-cancer activity of Anticanum in a cell culture was made in a culture of HeLa-2 cells. For this purpose, 20-120 mcg of MPs per ml of medium were added to the 199 medium. (See Table 2.) A culture without MPs in it was used as a control. Cultures were observed daily over the course of five days. The Minimum Active Dose (MAD) of Anticanum was also considered to be the minimum amount of the drug that caused a degeneration of 90-95% of the cells (Table 12).

TABLE 12
Comparative Sensitivity Characteristics
of Cultures of HeLa-2 Tumor Cells to MPs

| | | Anti-Cancerous Activity of | |
|---|---|---|---|
| | | MPs in Cell Culture | |
| Drug | MAD in mcg/ml | Control | Experiment |
| MPs | 120 | 0 | ++++ |
| Taxotere | 10 | 0 | ++ |
| Physical Solution | — | 0 | 0 |

1 Cytopathic activity;
++++ degeneration of 100% of the cells
0 lack of degeneration.

In establishing the minimum concentration of MPs that will slow the growth of cells, a comparison was made between the number of surviving cells and the concentration of MPs in the solution.

TABLE 13
The Effect of MPs on HeLa Cells

| | | Number of live cells | | Number of live | |
|---|---|---|---|---|---|
| | Number of cells | after incubation, | | cells after | |
| Dose, | before incubation, | millions, ±1000 | | incubation, % | |
| mcg/ml | millions | MP | Taxotere | MP | Taxotere |
| 20 | 151000 ± 1000 | 70000 | 140000 | 46 | 93 |
| 40 | 152000 ± 1000 | 37200 | 138000 | 24 | 91 |
| 60 | 151000 ± 1200 | 16200 | 1000 | 14 | 0.12 |
| 80 | 154000 ± 1000 | 0 | 127000 | 0 | 82 |
| 100 | 152000 ± 1000 | 0 | 140000 | 0 | 92 |
| 120 | 150000 ± 1000 | 0 | 152000 | 0 | 101 |

As may be seen in Table 13, an effective dose of MPs is between 80-120 mcg/ml solution.

MPs led to a 100% degeneration of tumor cells at a dose of 80-120 mcg/ml. To confirm the in vivo anti-tumor activity, MPs were studied in models of benzidine skin sarcoma and reinjected ascites adenocarcinomas in Barbados mice.

Study of the Anti-Cancer Activity of MPs on Benzidine Sarcoma.

Before applying it to the silica gel, 7 ml of a solution of 2% benzidine and 0.9% sodium chloride was added until an opalescent suspension was formed (1 g silica gel for 5 ml NaCl solution). Twenty-five Barbados mice of both sexes with a weight of 18-20 g that were kept on a vivarium diet were administered benzidine and phorbol acetate immobilized on silica gel subcutaneously near the neck every three days. After two weeks, 18 animals had developed tumors of different sizes in the form of a small bump on the neck near the silica gel granulomas. Each group of animals was administered the corresponding compound parenterally at a dose of 100 mcg/kg weight twice a day for two weeks, beginning at 16 days after administration of the carcinogen.

TABLE 14
A Comparison of the Anti-Tumor Activity of MPs
in Comparison to the Combination of an Analog
(Taxotere) and a Placebo (Physical Solution).

Weight of Animal (g)

| Drug Name | Before Treatment | After Treatment |
|---|---|---|
| Taxotere | 27 ± 2 | 23 ± 1.1 (2 mice died) |
| MPs | 26 ± 2 | 15 ± 1.5 |
| Physical Solution | 26 ± 2 | 38 ± 1.3 (5 mice died) |

Note:
n = 7, p > 0.05 in comparison with the control and previous data.

As may be seen in Table 14, MPs decreased the weight of the experimental animals by 11 g; the control animals' weight continued to increase, and some of them died. After the dissection of the silica gel granulomas, it was established that the animals treated with the MPs did not show signs that the granulomas had turned into malignant sarcomas.

The animals' survival rates are presented in Table 15.

TABLE 15
Survival Rates of Animals with Benzidine Skin Sarcoma

| Drug Name | Animal Survival, Days |
|---|---|
| Taxotere | 28 ± 1.1 |
| MP | 180 ± 5 |
| Physical Solution | 17 ± 0.9 |

Note:
n = 10, p > 0.05 in comparison with the control and previous data.

Thus the MPs prolong the life of animals more than ten times as long as does Taxotere.

A Study of the Anti-Tumor Activity of MPs on Ehrlich's Ascites Adenocarcinoma.

The anti-tumor activity of the compositions were studied in models of Ehrlich's ascites carcinoma in young Barbados mice of both sexes with weights between 15-17 g (70 individuals), which were kept on a vivarium diet.

50 mice were inoculated from a mouse with adenocarcinoma using an insulin syringe with 0.1 ml ascitic fluid in the region of the liver. Within seven days, 4 mice showed signs of tumors (the body weight and belly size increased); three mice died on the second day; one mouse did not show signs of a tumor.

15 mice were administered MPs intraperitoneally (see Table 16). 15 more mice were administered the MPs intravenously, and fifteen more were administered a 0.9% solution of sodium chloride.

TABLE 16
Quantitative Biological and Statistical Characteristics
in the Study of MPs Anti-Tumor Activity

| | | Time of Death of Animals | |
|---|---|---|---|
| | | after the First Injection | |
| | | Average Value | |
| | Form of | Days | |
| Substance | Introduction | Experimental Animals | Control Animals |
| MPs | IV | 48 ± 7 | 5 ± 2 |
| —//— | IP | 52 ± 8 | 5 ± 2 |
| Taxotere | IV | 15 ± 1 | 3 ± 1 |
| —//— | IP | 14 ± 1 | 3 ± 1 |

Note:
n = 10, p > 0.05 in comparison with the control and previous data.

MPs were given to those mice from which blood was drawn. Mice with Ehrlich's adenocarcinoma, after being given the tumor and treated, lived for 48-52 days when administered the modified substance, which is an average of 10 times longer than the control. At an accuracy level of more than 99.5%, we can confirm a significant increase in anticancer activity in MPs over the control, Taxotere. After dissection of the animals, signs of tumors and metastasis were not found in their bodies.

1 Feldherr C., Kallenbach E, Schultz N.//J.Cell.Biol.-1984.-Vol.99, P.2216-2222

2 Lanford R. E., Butel J. S. Cell.-1984.Vol.37., P. 801-813

3 Niederau C, Ferrell L. D., Grendell J. H. Caerulein-induced acute necrotizing pancreatitis in mice: protective effects of proglumide, benzotript, and secretin. Gastroenterology. 1985 May;88(5 Pt 1):1192-204.

1. A pharmaceutical composition, comprising a mixture of carboxylated oligopeptides, produced by enzymatic hydrolysis of natural polypeptides, that resulted in a oligopeptide mixture ranging of 2 to 30 amino acid residues, having free amino groups in the oligopeptide mixture and then carboxylated.

2. The composition of claim 1, wherein the natural polypeptides is a protein derived from eukaryotic cell.

3. The composition of claim 1, wherein the natural polynucleotide is a protein derived from procaryotic cell.

4. The composition of claim 1, wherein the enzymatic hydrolysis of the natural polypeptides is provided by protease.

5. The composition of claim 1, wherein the carboxylation of aminoacid residues with free amino groups is calculated through a mass modifier (Mm) on weighed portion of dry mixture of oligopeptides:
$$Mm = {M}_{r}^{m} \frac{n {2}^{( n - 1 )}}{( {2}^{n} - 1 )}$$
n- Quantity of the amino acid residues having free amino groups, available for modification of the taken weighed portion of dry oligopeptides composition
M^{m}_{I}- Molecular weight of the modifier g/mol.

6. The composition of claim 1, wherein the carboxylation of aminoacid residues is acylation by succinic anhydride.

7. The composition of claim 1, wherein the carboxylation of aminoacid residues is alkylation by monochloroacetic acid.
